(19) United States
(12) Patent Application Publication
(10) Pub. No.: US 2004/0172127 A1
(43) Pub. Date: Sep. 2, 2004

(54) Modular stent having polymer bridges at modular unit contact sites

(76) Inventor: John Kantor, Santa Rosa, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/731,968

(22) Filed: Dec. 9, 2003

Related U.S. Application Data
(60) Provisional application No. 60/432,278, filed on Dec. 9, 2002.

(51) Int. Cl. A61F 2/06

(52) U.S. Cl. 623/1.16; 623/1.46

(57) ABSTRACT

A radially expandable modular stent for implantation within the body of a patient is disclosed. The modular stent includes a first stent module defining a first passageway, at least a second stent module defining at least a second passageway, and a least one polymer bridge in communication with the first stent module and the second stent module. The polymer bridge couples the first stent module to the second stent module such that the first passageway and the second passageway are in fluid communication.

### RELATED APPLICATION

The present application claims priority to U.S. Provisional Application Serial No. 60/432,278 filed Dec. 9, 2002 the entire contents of which are herein incorporated by references in their entirety.

### FIELD OF THE INVENTION

The present invention relates to vascular implants, specifically vascular implants made using a modular construction. More specifically, the present invention related to radially expandable modular stents having modular bridges with polymer coatings thereon.

### BACKGROUND OF THE INVENTION

Stenosis is the narrowing of a lumen or an opening that occurs in organs, vessels, or other luminal structures within the body. A number of physiological complications have been associated with stenosis, such as ischemia cardiomyopathy, angina pectoris, and myocardial infarction. In response, several procedures have been developed for treating stenosis. For example, dilation, ablation, atherectomy, or laser treatments have been used to successfully treat luminal structures and improve the patency of stenotic lumens or openings. Typically, these procedures require the introduction of catheters, guide wires, stents, sheaths, or tubes into the stenotic lumen or opening prior to, during, and following the procedure. While these procedures have proven successful in treating stenosis in the past, several shortcoming associated with these procedures have been identified. For example, the insertion of these foreign materials into the luminal structure may lead to complications such as luminal scaring and restenosis. The occurrence of these complications following these procedures may depend on a variety of factors, including, for example, vessel location, vessel elasticity, lesion length, severity of injury, and an individual's wound healing propensities.

Typically, restenosis occurs in thirty to forty percent of the patients that undergo percutaneous transluminal coronary angioplasty (PTCA). Restenosis, which may be attributable to hyperproliferation of vascular smooth muscle cells and excess epithelialization, may result in the narrowing of the luminal structures. As a result, restenosis may be treated by a number of highly invasive surgical procedures such as coronary artery bypass graft surgery (CABG). While CABG procedures have proven useful in treating restenosis, several shortcoming have been identified. For example, the highly invasive nature of CABG procedure results in increased patient suffering as well as increased mortality rates. As a result, a number of less invasive procedures have been developed to treat restenosis.

One less invasive approach to treating restenosis involves the implantation of a radially expandable stent into the luminal structure. Stents are mechanical scaffoldings which may be inserted into an occluded region of a lumen or luminal structure to provide and maintain patency. During implantation, a stent is positioned on a delivery device such as a balloon catheter and advanced from a external location through a luminal pathway to an area of occlusion within the body of the patient. Thereafter, the delivery device may be actuated to deploy the radially expandable stent. Expansion of the radially expandable stent results in the application of force to the internal wall of the luminal structure, thereby improving the patency of the luminal structure. Thereafter, the delivery device may be removed from the patient's body.

Stents may be manufactured in a variety of lengths and diameters from a variety of materials ranging from metallic materials to biocompatible polymers and may incorporate therapeutic agents or medicaments. As a result, these drug eluding stents enabling the localized delivery of medicinal agents to a target site while providing radial support to the adjacent luminal structure.

In one embodiment, stents are manufactured by laser welding a series of stent modules or sections together thereby forming a unitary modular stent. Modular stent designs offer several advantages over other stent designs, including, for example, improved manufacturability, improved stent flexibility, and the ability of the surgeon to customize the stent architecture depending on intended use. However, module compaction has been identified as one shortcoming associated with current modular stent designs. Module compaction, otherwise known as “train wrecking,” arises when one or more stent modules of a modular stent are longitudinally compressed during implantation. As a result, the compressed stent modules may fail to apply sufficient radially expanding force to the luminal structure. In addition, flow through the internal passageway of the stent may be reduced.

In light of the foregoing, there is an ongoing need for a radially expandable modular stent capable of providing sufficient radially expanding force to a luminal structure while having a decreased potential of module compaction.

### BRIEF SUMMARY OF THE INVENTION

In one embodiment, a radially expandable modular stent is disclosed. The modular stent includes a first stent module defining a first passageway, at least a second stent module defining at least a second passageway, and a least one polymer bridge in communication with the first stent module and the second stent module. The polymer bridge couples the first stent module to the second stent module such that the first passageway and the second passageway are in fluid communication.

In another embodiment, a coated radially expandable modular stent is disclosed. The coated modular stent comprises a first stent module defining a first passageway, at least a second stent module defining at least a second passageway, and a least one polymer bridge coating the first stent module and the second stent module. The polymer bridge couples the first stent module to the second stent module such that the first passageway and the second passageway are in fluid communication.

In yet another embodiment, a spot-bridged radially expandable modular stent is disclosed. The spot-bridged modular stent comprises a first stent module defining a first passageway, at least a second stent module defining at least a second passageway, and a least one polymer bridge in communication with the first stent module and the second stent module. The polymer bridge may be positioned at a point of contact of between the first stent module and the second stent module such that the polymer bridge couples the first stent module to the second stent module wherein the first passageway and the second passageway are in fluid communication.

In addition, a method of making a radially expandable modular stent is disclosed and includes forming a first stent module from at least one stent material, forming at least a second stent module from the at least one stent material, and coupling the second stent module to the first stent module with a polymer bridge.

Other objects, features, and advantages of the present invention will become apparent from a consideration of the following detailed description.

### BRIEF DESCRIPTION OF THE DRAWINGS

The modular stent having polymer bridges at modular unit contact sites will be explained in more detail by way of the accompanying drawings, wherein:

FIG. 1 shows a perspective view of a modular stent having a polymer bridge coupling two stent modules together;

FIG. 2 shows a perspective view of a stent module of the modular stent;

FIG. 3 shows a cross-sectional view of a modular stent having a polymer bridge forming a polymer hinge between two stent modules;

FIG. 4 shows a cross-sectional view of a modular stent having a polymer bridge forming a polymer weld coupling two stent modules together;

FIG. 5 shows a cross-sectional view of another embodiment of a modular stent having a polymer bridge forming a polymer coupler coupling two stent modules together;

FIG. 6 shows a cross-sectional view of another embodiment of a modular stent having a polymer bridge forming a polymer spot weld coupling two stent modules together; and

FIG. 7 shows a perspective view of a modular stent having a polymer bridge forming a polymer coupler coupling two stent modules together.

FIG. 8 shows a side view of a modular stent positioned on a balloon catheter;

FIG. 9 shows a side view of an unexpanded modular stent positioned on a deflated expandable balloon of a balloon catheter; and

FIG. 10 shows a side view of an expanded modular stent positioned on a inflated expandable balloon of a balloon catheter.

### DETAILED DESCRIPTION OF THE INVENTION

The following detailed description and the accompanying drawings are intended to describe and show certain presently preferred embodiments of the present invention, and are not intended to limit the scope of the present invention in any way.

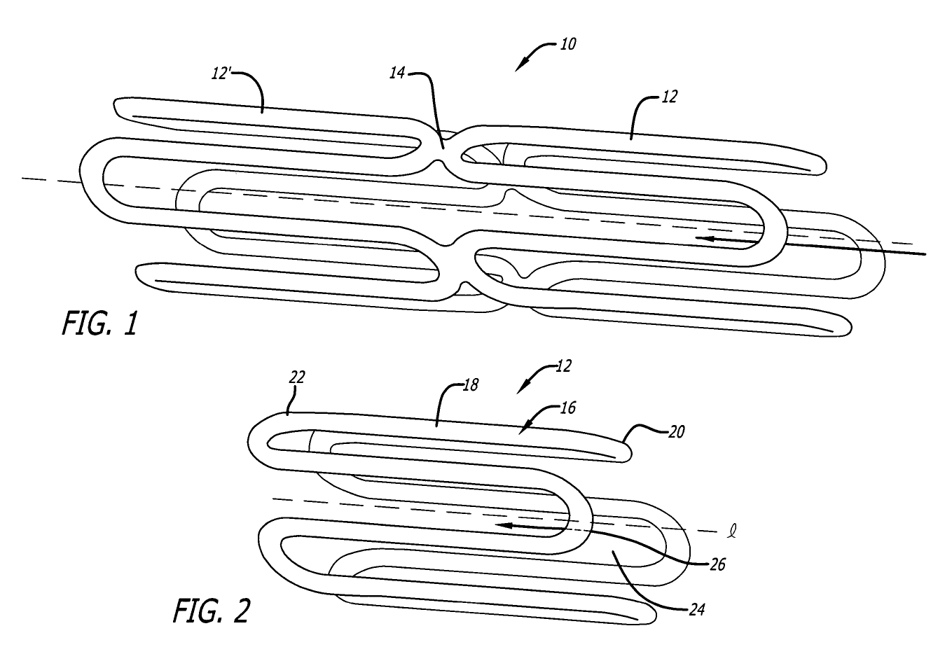

FIG. 1 shows an embodiment of the radially expandable modular stent having polymer bridges at modular unit contact sites. As shown in FIG. 1, the radially expandable modular stent 10 comprises at least two stent modules 12, 12′ joined by at least one polymer bridge 14. Those skilled in the art will appreciate that the radially expandable modular stent 10 may be manufactured in a variety of sizes, lengths, and diameters (inside diameters as well as outside diameters). In one embodiment, the stent 10 may be manufactured having a length of 2 mm to 60 mm and and having an outside diameter of 0.05 mm to 0.80 mm, thereby permitting the use of the stent 10 within the patient's coronary artery or related vascular structures. Furthermore, the radially expandable stent 10 may be manufactured from a plurality of materials, including, without limitation, stainless steel, tantalum, titanium, Nickel-Titanium alloys, shape memory alloys, super elastic alloys, low-modulus Ti—Nb—Zr alloys, colbalt-nickel alloy steel (MP-35N), various biologically compatible polymers and elastomers, including non-porous, porous, and microporous polymers or elastomers. In an alternate embodiment, the radially exapandable modular stent 10 may be coated with or have applied thereto at least one therapeutic agent or medicament, thereby enabling the radially expandable modular stent 10 to elude or deliver at least one therapeutic agent or medicament to target site within the body of a patient. The term “agent” or “drug” as used herein means any compound intended for use in animals having a desired effect. Non-limiting examples include anticoagulants, such as an RGD peptide-containing compound, heparin, antithrombin compounds, platelet receptor antagonists, anti-thrombin antibodies, anti-platelet receptor antibodies, aspirin, protaglandin inhibitors, platelet inhibitors, or tick anti-platelet peptide. Other classes of agents include vascular cell antiproliferative agents, such as a growth factor inhibitor, growth factor receptor antagonists, transcriptional repressor or translational repressor, antisense DNA, antisense RNA, replication inhibitor, inhibitory antibodies, antibodies directed against growth factors, cytotoxic agents, cytoskeleton inhibitors, peroxisome proliferator-activated receptor gamma (PPAR_{Y}) agonists, molecular chaperone inhibitors and bifunctional molecules. The agent can also include cholesterol-lowering agents, vasodilating agents, and agents which interfere with endogenous vasoactive mechanisms. Other examples of agents can include anti-inflammatory agents, anti-platelet or fibrinolytic agents, anti-neoplastic agents, anti-allergic agents, anti-rejection agents, metaloprotease inhibitors, anti-microbial or anti-bacterial or anti-viral agents, hormones, vasoactive substances (including vasodilators), anti-invasive factors, anti-cancer drugs, antibodies and lymphokines, anti-angiogenic agents, radioactive agents and gene therapy drugs, among others.

Specific non-limiting examples of drug agents that fall under one or more of the above categories include paclitaxel, docetaxel and derivatives, epothilones, nitric oxide release agents, heparin, aspirin, coumadin, D-phenylalanyl-prolyl-arginine chloromethylketone (PPACK), hirudin, polypeptide from angiostatin and endostatin, benzoquinone ansamycins including geldanamycin, herbimycin and macbecin, methotrexate, 5-fluorouracil, estradiol, P-selectin Glycoprotein ligand-1 chimera, abciximab, exochelin, eleutherobin and sarcodictyin, fludarabine, sirolimus, rapamycin, ABT-578, certican, Sulindac, tranilast, thiazolidinediones including rosiglitazone, troglitazone, pioglitazone, darglitazone and englitazone, tetracyclines, VEGF, transforming growth factor (TGF)-beta, insulin-like growth factor (IGF), platelet derived growth factor (PDGF), fibroblast growth factor (FGF), RGD peptide, estrogens including 17 beta-estradiol and beta or gamma ray emitter (radioactive) agents, vasodilators such as nitric oxide (NO), various marking agents including radio-opaque or echogenic materials and combinations thereof.

FIG. 2 shows an exemplary stent module 12 forming at least one section of the radially expandable modular stent 10 (see FIG. 1). As shown, the stent module 12 comprises a body member 16 having a generally linear body section 18 positioned between a first end 20 and a second end 22. In the illustrated embodiment, the body member 16 comprises a sinusoidal body member, although those skilled in the art will appreciate that any modular stent architecture could be used. One or more body openings 24 may be formed within the sinusoidal body member 16. In addition, a passageway 26 may be formed by the sinusoidal body member 16. In the illustrated embodiment, the passagway 26 is positioned along the longitudinal axis I of the stent module 12. Those skilled in the art will appreciate that the modular stent 10 of the present invention may be manufactured in a variety of architectures and orientations as known in the art. Furthermore, any number of stent modules 12 may be joined or coupled depending on the physiological constraints of the patient. In one embodiment, any number of stent modules 12 of equal length and/or diameter may be coupled together to form the modular stent shown in FIG. 1. In an alternate embodiment, any number of stent modules 12 of unequal length and/or diameter may be coupled together to form the modular stent shown in FIG. 1. Furthermore, stent modules 12 manufactured from the same or different materials or coated with the same or different therapeutic agents may be coupled together.

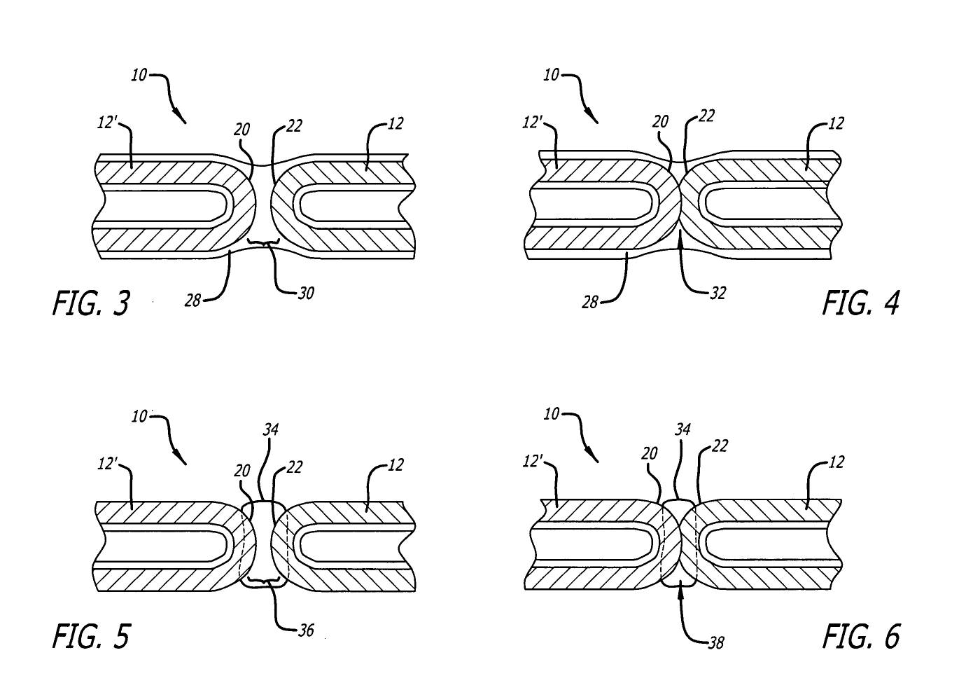

FIGS. 34 show an embodiment of the modular stent 10 wherein the stent modules 12, 12′ are coated with a polymer material thereby coupling the stent modules 12, 12′ together. FIG. 3 shows one embodiment of the modular stent 10 having the second end 22 of a stent module 12 coupled to a first end 20 of another stent module 12′. As shown, the stent modules 12, 12′ may be coated with a polymer thereby forming a polymer bridge 28 between the stent modules 12, 12′. A flexible hinge or gap 30 may be formed between the second end 22 of the stent module 12 and the first end 20 the stent module 12′, thereby permitting movement of the stent modules 12, 12′ relative to each other and enhancing the lateral and longitudinal flexibility of the stent 10. FIG. 4 shows an alternate embodiment of the stent 10 wherein the second end 22 of a stent module 12 may be coupled to and in contact with the first end 20 of another stent module 12′, thereby forming a polymer weld 32 there between. As shown, the stent modules 12, 12′ may be coated with a polymer thereby forming a polymer bridge 28 coupling the stent modules 12, 12′ together. Those skilled in the art will appreciate that any number of stent modules may be coupled together to form a modular stent.

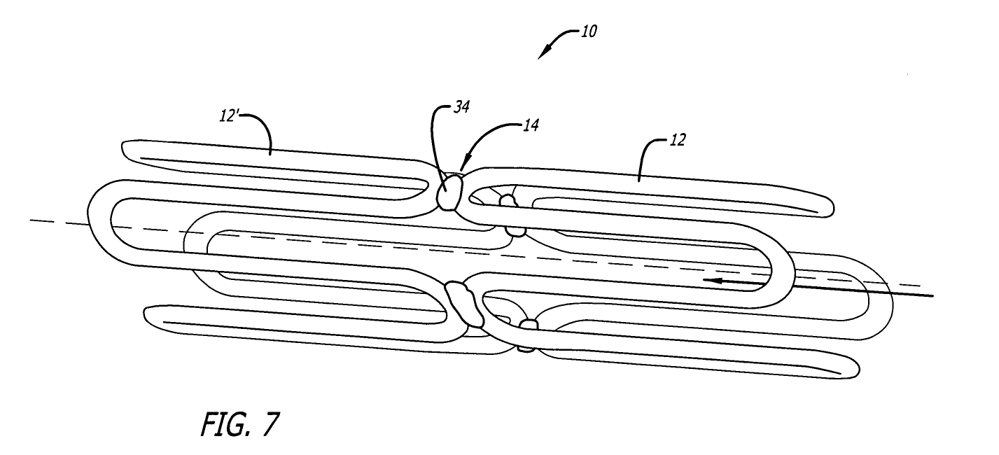

FIGS. 5-7 show an alternate embodiment of the modular stent 10 wherein a polymer coupler 34 is used to couple the stent modules 12, 12′ together. FIG. 5 shows the modular stent 10 having the second end 22 of a stent module 12 coupled to a first end 20 of another stent module 12′. As shown, a polymer coupler 34 is positioned between the stent modules 12, 12′ thereby forming a polymer bridge 28 between the stent modules 12, 12′. A flexible hinge or gap 36 may be formed between the second end 22 of the stent module 12 and the first end 20 the stent module 12′, thereby permitting movement of the stent modules 12, 12′ relative to each other and enhancing stent flexibility. FIG. 6 shows an alternate embodiment of the stent 10 wherein the second end 22 of a stent module 12 may be coupled to and in contact with the first end 20 of another stent module 12′, thereby forming a polymer spot weld 38 there between. As illustrated in FIGS. 5-7, the polymer coupler 34 may be applied to the stent modules 12, 12′ only at a point of contact with another stent module. In the illustrated embodiment, the polymer coupler 34 is applied to the ends of the stent modules 12, although the polymer coupler 34 may be applied anywhere along the body of the stent modules as desired by the user. Furthermore, those skilled in the art will appreciate that any number of stent modules may be coupled together to form a modular stent. FIG. 7 shows a modular stent 10 having polymer couplers 34 forming the polymer bridge 14 which couples the stent modules 12, 12′ together.

Referring again to FIGS. 1 and 7, the polymer bridge 14 may be manufactured from a variety of biologically-compatible materials. For example, in one embodiment, at least one polymer bridge 14 may be manufactured from a bioabsorbable polymer material. Exemplary bioabsorbable polymer material may include, without limitation, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, poly-N-alkylacrylamides, poly depsi-peptide carbonate, and polyethylene-oxide based ployesters.

In an alternate embodiment, at least one polymer bridge 14 is manufactured from a biostable polymer material having a relatively low chronic tissue response. Exemplary biostable polymer materials include, for example, polyurethanes, silicones, and polyesters. Other biostable polymer materials could also be used if the biostable polymer material can be dissolved and cured or polymerized on a medical device, and may include polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl chloride, polyvinyl ethers, polyvinyl methyl ether, polyvinylidene halides, polyvinylidene fluoride, polyvinylidene chloride, polyacrylonitrile, polyvinyl ketones, polyvinyl aromatics, polystyrene, polyvinyl esters, polyvinyl acetate, copolymers of vinyl monomers with each other and olefins, ethylene-methyl methacrylate copolymers, acrylonitrile-styrene copolymers, ABS resins, ethylene-vinyl acetate copolymers, polyamides, Nylon 66, polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, or various combinations thereof. In one embodiment, at least one polymer bridge 14 is manufactured from a bioabsorbable polymer material while at least one other polymer bridge 14 is manufactured from a biostable polymer material. In another embodiment, the polymer bridge 14 may be biodegradable and may permit the polymer bridge 14 to degrade over time thereby leaving several individual radially expandable stent modules 12, 12′ positioned within a luminal structure. In an alternate embodiment, the modular stent 10 of the present invention in further include a stent graft. For example, at least one drug eluding stent graft may be positioned on the external surface, the internal surface, of both surfaces of the stent 10.

Those skilled in the art will appreciate that the modular stent 10 may be manufactured in a variety of ways. For example, individual stent modules 12, 12′ may be formed by laser cuffing a colbalt-nickel alloy steel (MP-35N) tube of a desired diameter to a desired length. In an alternate embodiment, the individual stent modules may be manufactured by deforming a ring of stent material. For instance, the ring may be manufactured from colbalt-nickel alloy steel (MP-35N). Thereafter, the body openings 24 (see FIG. 2) may be formed in the tube. In an illustrative embodiment, the body openings 24 may be laser cut into the tube thereby forming a stent module. Any number of stent modules may be positioned on a mandrel or other stent module positioning device in preparation to receive the polymer bridge material. The polymer bridge 14 may be applied to at least one surface of the stent modules 12, 12′ in a variety of ways, including, for example, dipped, sprayed, or vapor deposited. If desired, a therapeutic agent may be applied to the stent modules 12, 12′ prior to, during, of following the application of the polymer bridge material.

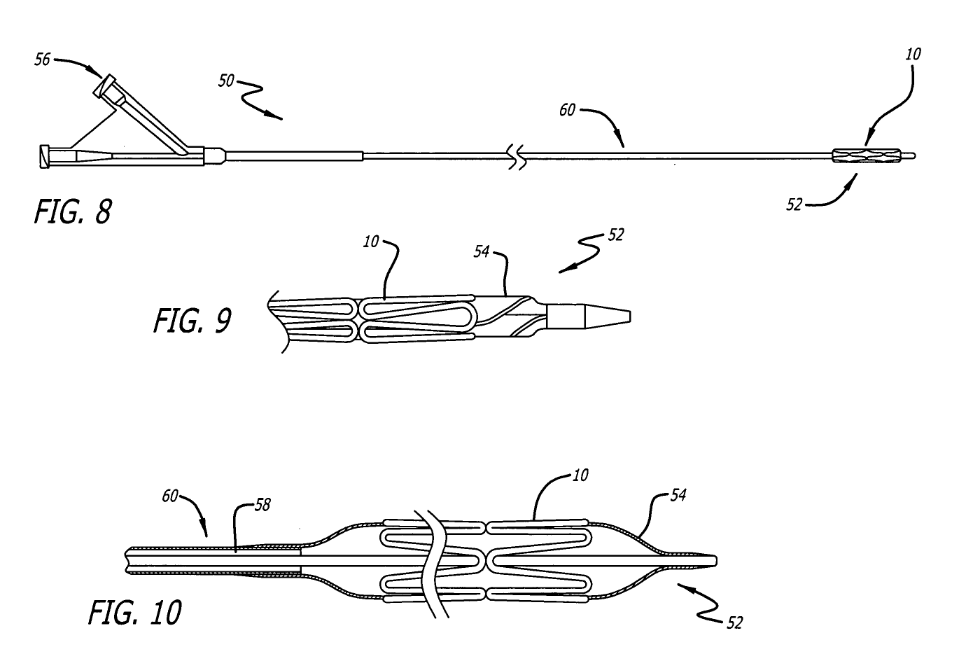

The modular stent 10 may be delivered to an area of interest within the body of a patient using a variety of techniques known in the art. For example, FIGS. 8-10 show the modular stent 10 positioned on a balloon catheter 50. As shown, the balloon catheter 50 includes a distal portion 52 disposing a expandable balloon body 54. The expandable balloon body 54 is in communication with an inflation port 56 through an actuation lumen 58 formed within the elongated body 60 of the balloon catheter 50. As shown in FIGS. 8 and 9, prior to the implantation of the modular stent 10 the expandable balloon 54 is deflated. As a result, the modular stent 10 is positioned proximate to the elongated body 60. The distal portion 52 of the balloon catheter 50 is inserted into the vascular structure of the patient and advanced trough a circulatory pathway to a position proximate an area of interest. Thereafter, an inflation fluid such as saline solution is introduced into the expandable balloon 54 though the inflation port 56, thereby resulting in the radial expansion of the expandable balloon 54. As shown in FIG. 10, the radially expandable modular stent 10 expands in response thereto resulting in the application of the modular stent 10 to the area of interest. Once the modular stent 10 is applied to the area of interest, the inflation fluid is evacuated from the expandable balloon thereby resulting in the deflation thereof. The balloon catheter is retracted through the circulatory pathway and the insertion wound in the patient is closed.

In an alternate embodiment the radially expandable modular stent 10 of the present invention is provided with a polymer coating. Polymer coatings are useful in increasing bare-metal stent biocompatibility and in serving as reservoirs for eluteable bioactive agents (drugs). Many different polymers are known to be useful as coatings for implantable medical devices and the state-of-the-art in controlled release coatings for medical devices has increased rapidly in the last decade. However, polymer selection must still be tailored to the specific type of medical device. For example, in the present invention the medical device is a vascular stent intended for implantation within a hemodynamic environment.

Stents made in accordance with the present invention mare flexible and subject to expansive forces n addition to twisting and bending. Consequently the polymer coatings must be able to sustain flexion forces, be biocompatible and adhere well to the stent surface in order to minimize luminal wall irritation and prevent thrombosis. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

When used as a drug delivery platform the coating of the present invention are combined with a drug in a fashion optimally suited to deliver the drug, or drugs, over a predetermined time and specific kinetic profile. For example, in some embodiments a burst of drug is desired immediately after stent placement followed by a slower, more sustained release profile. Yet, in other applications an initial burst of drug may be undesirable. Consequently, it is necessary to adjust the polymer-to-drug ratio, among other parameters, in order to achieve the desired drug release characteristics.

In the present invention a first polymer coating for the modular bridges can provide a first drug eluting polymer and the polymer coating provides a second polymer coating. As will be discussed further below, this unique confirmation featuring a first polymer associated with the stent's mechanical structure and a second polymer serving as a structural covering provides controlled release vascular device of great versatility.

The polymer-to-drug ratio will depend on the drug's interactions with the polymer. In one embodiment of the present invention a nitric oxide releasing polymer may be used to form a highly anti-thrombogenic polymer topcoat and a prolonged anti-restenotic such as an anti-proliferative compound used in the polymer bridge. After implanting the stent NO will be released for a predetermined time followed by prolonged delivery of the anti-restenotic. The present inventor also envisions other combinations. For example, a first higher level of anti-proliferative can be evenly dispersed near the surface of the coating polymer so that a burst of ant-restenotic drug is delivered following stent implantation. A second lower does of the same or different anti-proliferative can then be released more slowly at lower concentrations from the polymer coating the bridges underlying the polymer top coat.

In yet another embodiment, the same anti-restenotic drug can be used in both the topcoat polymer and the bridge polymer. However, polymers with different solubility parameters are used such that the drug is released at different rates and over different time periods. In another embodiment the topcoat acts as a gate-keeping controlled release barrier in synergy with the release rates of the underlying bridge polymer.

Other physical factors that contribute to polymer-to-drug ratios include the polymer coating thickness, the number of layers, the presence or absence of a primer coat over the stent and the size of the medical device to be coated. In the case of the present invention, in one embodiment a first anti-retenotic drug is incorporated into the polymer bridge and a second anti-restenotic drug is incorporated into the coating polymer. Any number of drug comminations are envisioned and it is not intended that merely two different drugs be employed, rather any number of drugs may be used. A wide ratio of therapeutic substance-to-polymer could therefore be appropriate and could range from about 0.1% to 99% by weight of therapeutic substance-to-polymer.

In closing it is understood that the embodiments of the invention disclosed herein are illustrative of the principles of the invention. Accordingly, the present invention is not limited to that precisely as shown and described in the present invention.

What is claimed is:

1. A radially expandable modular stent for implantation within the body of a patient, comprising:
a first stent module defining a first passageway;
at least a second stent module defining at least a second passageway; and
a least one polymer bridge in communication with said first stent module and at least said second stent module, said polymer bridge coupling said first stent module to at least said second stent module wherein said first passageway and said at least said second passageway are in fluid communication.

2. The apparatus of claim 1 wherein said polymer bridge comprises a polymer material applied to at least one surface of said first stent module and at least said second stent module.

3. The apparatus of claim 2 wherein said polymer material is applied to an external surface of said first stent module and at least said second stent module.

4. The apparatus of claim 2 wherein said polymer material is applied to an internal surface of said first stent module and at least said second stent module.

5. The apparatus of claim 2 wherein said polymer material is applied to an internal surface and an external surface of said first stent module and at least said second stent module.

6. The apparatus of claim 2 wherein said polymer material is applied to at least said second stent module at a point of contact with said first stent module.

7. The apparatus of claim 1 wherein said polymer bridge further comprises a polymer hinge defining a gap between said first stent module and at least said second stent module.

8. The apparatus of claim 1 wherein said polymer bridge further comprises a polymer weld coupling at least said second stent module to said first stent module, wherein at least said second stent module is in contact with said first stent module.

9. The apparatus of claim 1 wherein at least one said polymer bridge manufactured from a biologically compatible polymer.

10. The apparatus of claim 9 wherein said biologically compatible polymer is selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, biomolecules, fibrin, fibrinogen, cellulose, starch, collagen, hyaluronic acid, polyurethanes, silicones, polyesters, polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl chloride, polyvinyl ethers, polyvinyl methyl ether, polyvinylidene halides, polyvinylidene fluoride, polyvinylidene chloride, polyacrylonitrile, polyvinyl ketones, polyvinyl aromatics, polystyrene, polyvinyl esters, polyvinyl acetate, copolymers of vinyl monomers, ethylene-methyl methacrylate copolymers, acrylonitrile-styrene copolymers, ABS resins, ethylene-vinyl acetate copolymers, polyamides, Nylon 66, polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

11. The apparatus of claim 1 wherein said polymer bridge is biologically degradable.

12. The apparatus of claim 1 wherein at least one of said first stent module, said second stent module, and said polymer bridge includes at least one therapeutic agent selected from the group consisting of polytetrafluoroethylene, anti-thrombotic agents, platlet-derived growth factor (PDGF), tranforming growth factor-beta (TGF-beta), heparin, anti-inflamatory agents, anti-proliferation agents, rapamycin, angiopeptin, methotrexate, paclitaxel, anti-microbial agents, anti-metabolic agents, anti-platlet agents, anti-coagulant agents, Nitric Oxide releasing agents, chaperone inhibitors, geldanamycin, glitazones, metalloproteinase inhibitors (MMPI), antisense polynucleotides, and transforming nucleotides.

13. The apparatus of claim 1 wherein at least one of said first stent module, said second stent module, and said polymer bridge includes at least one radio-opaque or echogenic material.

14. The apparatus of claim 1 wherein at least one of said first module and at least said second stent module is manufactured from at least one material selected from the group consisting of stainless steel, tantalum, titanium, Nickel-Titanium alloys, shape memory alloys, super elastic alloys, low-modulus Ti—Nb—Zr alloys, colbalt-nickel alloy steel (MP-35N), biologically compatible polymers, and biologically compatible elastomers.

15. The apparatus of claim 1 wherein at least one of said first module and at least said second stent module is porous.

16. The apparatus of claim 1 wherein at least one of said first module and at least said second stent module is non-porous.

17. A radially expandable modular stent for implantation within the body of a patient, comprising:
a first stent module defining a first passageway;
at least a second stent module defining at least a second passageway; and
at least one polymer bridge coating said first stent module and at least said second stent module, said polymer bridge coupling said first stent module to at least said second stent module wherein said first passageway and said at least said second passageway are in fluid communication.

18. A radially expandable modular stent for implantation within the body of a patient, comprising:
a first stent module defining a first passageway;
at least a second stent module defining at least a second passageway; and
a least one polymer bridge positioned at a point of contact of said first stent module and at least said second stent module, said polymer bridge coupling said first stent module to at least said second stent module wherein said first passageway and said at least said second passageway are in fluid communication.

19. A method of making a radially expandable modular stent, comprising:
forming a first stent module from at least one stent material;
forming at least a second stent module from said at least one stent material; and
coupling at least said second stent module to said first stent module with a polymer bridge.

20. The method of claim 19 wherein further comprising coating at least one surface of said first stent module and at least said second stent module with a polymer material to form said polymer bridge.

21. The method of claim 20 further comprising coating an external surface of said first stent module and at least said second stent module with a polymer material to form said polymer bridge.

22. The method of claim 20 further comprising coating an internal surface of said first stent module and at least said second stent module with a polymer material to form said polymer bridge.

23. The method of claim 20 further comprising coating an external surface and an internal surface of said first stent module and at least said second stent module with a polymer material to form said polymer bridge.

24. The method of claim 19 further comprising applying a polymer material to at least said second stent module and to said first stent module at a location wherein said second stent module contacts said first stent module.

25. The method of claim 19 wherein said polymer bridge is applied to said first stent module and at least said second stent module by at least one process selected from the group consisting of dipping, spraying, and vapor deposition.

26. The method of claim 19 further comprising applying at least one therapeutic agent to at least said first stent module, said second stent module, and said polymer coating.

27. The method of claim 27 further coating at least one of said first stent module, said second stent module with at least one therapeutic agent selected from the group consisting of anti-thrombotic agents, platlet-derived growth factor (PDGF), tranforming growth factor-beta (TOF-beta), heparin, anti-inflamatory agents, anti-proliferation agents, rapamycin, angiopeptin, methotrexate, paclitaxel, anti-microbial agents, anti-metabolic agents, anti-platlet agents, anti-coagulant agents, Nitric Oxide releasing agents, chaperone inhibitors, geldanamycin, glitazones, metalloproteinase inhibitors (MMPI), antisense polynucleotides, and transforming nucleotides.

28. A vascular device a plurality of stent modules comprising:
a first stent module defining a first passageway;
at least a second stent module defining at least a second passageway; and
a least one bridge comprising a first polymer in communication with said first stent module and at least said second stent module;
a coating comprising a second polymer covering said plurality of stent modules wherein said first and said second polymer include at least one drug.

29. The vascular device according to claim 28 wherein said first and second polymer are the same polymer.

30. The vascular device according to claim 28 wherein said first and second polymer are different polymers.

31. The vascular device according to any one of claims 28 through 30 wherein said drug is selected from the group consisting of paclitaxel, docetaxel and derivatives, epothilones, nitric oxide release agents, heparin, aspirin, coumadin, D-phenylalanyl-prolyl-arginine chloromethylketone (PPACK), hirudin, polypeptide from angiostatin and endostatin, benzoquinone ansamycins including geldanamycin, herbimycin and macbecin, methotrexate, 5-fluorouracil, estradiol, P-selectin Glycoprotein ligand-1 chimera, abciximab, exochelin, eleutherobin and sarcodictyin, fludarabine, sirolimus, rapamycin, ABT-578, certican, Sulindac, tranilast, thiazolidinediones including rosiglitazone, troglitazone, pioglitazone, darglitazone and englitazone, tetracyclines, VEGF, transforming growth factor (TGF)-beta, insulin-like growth factor (IGF), platelet derived growth factor (PDGF), fibroblast growth factor (FGF), RGD peptide, estrogens including 17 beta-estradiol and beta or gamma ray emitter (radioactive) agents, vasodilators such as nitric oxide (NO), various marking agents including radio-opaque or echogenic materials and combinations thereof.
